(19) United States
(12) Patent Application Publication
(10) Pub. No.: US 2025/0059210 A1
(43) Pub. Date: Feb. 20, 2025

(54) SYNTHESIS OF METAL ORGANIC FRAMEWORK (MOF) MATERIALS WITH HIGH ADSORPTION CAPACITY OF ORGANIC COMPOUNDS AND CO2 CAPTURE

(71) Applicants: UNIVERSITE DE STRASBOURG, STRASBOURG (FR); CENTRE NATIONAL DE LA RECHERCHE SCIENTIFIQUE, PARIS (FR)

(72) Inventors: Benoît LOUIS, GEISPOLSHEIM (FR); Nizami ISRAFILOV, STRASBOURG (FR); Jean-Marc PLANEIX, STRASBOURG (FR)

(21) Appl. No.: 18/721,638

(22) PCT Filed: Dec. 22, 2022
(86) PCT No.: PCT/EP2022/087509

§ 371 Date: Jun. 18, 2024

(30) Foreign Application Priority Data
Dec. 22, 2021 (EP) 21306902.4

(51) Int. Cl. C07F 1/08; C07F 3/02; C07F 3/06; C07F 7/00

(52) U.S. Cl. CPC C07F 1/08; C07F 3/02; C07F 3/06; C07F 7/00

(57) ABSTRACT

Metal-organic framework material (MOF), comprising at least one metal center and at least one organic compound selected among the compounds according to formula (I):

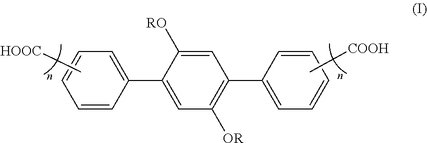

wherein n is from 1 to 2,
R is —(CH₂—CH₂—O)ₘ—CH₃ with m from 1 to 7;
preferably when n=1, the two —COOH groups are located in the para position relatively to the aryl substituted by the two RO groups; and
preferably when n=2, the four —COOH groups are located in the meta position relatively to the aryl substituted by the two RO groups.

### FIELD OF THE INVENTION

The present invention relates to aryl compounds comprising carboxylate groups and functionalized with ether groups, and to their association with metals in order to obtain crystalline solid materials, and more particularly to obtain metal organic frameworks or MOFs.

In what follows, the numbers between brackets ([]) refer to the List of References provided at the end of the document.

### BACKGROUND OF THE INVENTION

The use of amine-based liquids for carbon dioxide (CO₂) capture and storage is well known. Nevertheless, these liquids are toxic, corrosive and require a lot of energy to release the adsorbed CO₂ for regeneration, the search for an alternative has attracted much interest.

The use of metal organic frameworks (MOFs) compounds has been widely investigated for the capture of small molecules such as CO₂. Metal-organic frameworks (MOFs), also known as porous coordination polymers (PCPs) or coordination networks, are crystalline materials that can be easily self-assembled from metal ions or metal clusters with organic ligands. These MOFs are already extensively used in industry for the preparation of a large scale of materials, and they are particularly convenient for the preparation of new materials as MOFs can be processed and functionalized to impart new physical and chemical properties, charge conduction, and adjustable porosity.

MOF materials are solid adsorbents that are neither corrosive nor toxic and can be easily regenerated by known pressure swing adsorption techniques, or temperature swing adsorption techniques. Besides MOFs are porous coordination polymers, having an inorganic-organic hybrid framework that comprises metal ions and organic ligands coordinated to the metal ions. These materials are organized into one-, two-or three-dimensional lattices, in which the metallic species are joined together periodically by spacer ligands. The framework of these solids has both inorganic parts and organic parts, the cavities of which may be occupied by water molecules or by organic molecules that are easy to extract without deterioration of the backbone.

The use of aryl ligands incorporated into the MOF as the main component of its organic part has been investigated for the absorption of small molecules such as H₂ or CO₂.

Reference [1] describes terphenyl-tetracarboxylate ligands with alkoxy functions containing alkyl side chains for the synthesis of MOFs for H₂ adsorption. Besides, in [2] other terphenyl-tetracarboxylate ligands with alkoxy functions containing alkyl side chains for the synthesis of MOFs are reported for the adsorption of CO₂, H₂, N₂ and methane. There remains a need for the development of new synthetic processes which allow the preparation of MOF exhibiting enhanced properties of CO₂ adsorption or other organic and/or inorganic molecules such as dye, and with properties that outperform those of all the reported MOFs so far in this respect.

### BRIEF DESCRIPTION OF THE INVENTION

The present invention relates to an organic compound for the preparation of a MOF, said organic compound being selected among the compounds according to formula (I):

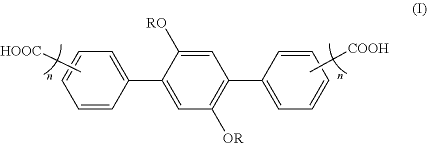

wherein n is from 1 to 2,
R is —(CH₂—CH₂-O)ₘ-CH₃ with m from 1 to 7;
preferably when n=1, the two —COOH groups are located in the para position relatively to the aryl substituted by the two RO groups; and
preferably when n=2, the four —COOH groups are located in the meta position relatively to the aryl substituted by the two RO groups.

The inventors have demonstrated unexpectedly that MOF comprising ligands consisting of those organic compounds provides hight stability in water, with great thermostability as they are stable up to 300° C. and even under water steam, and that CO₂ absorption or dye molecules such as methylene blue was better compared to the MOFs described in the previous art.

Advantageously, n is 2, the organic compound being selected among the compounds according to formula (II):

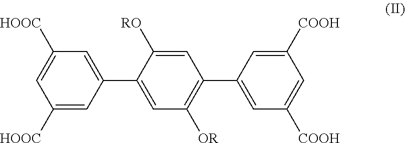

and preferably, m is less than 4.

The present invention relates also to a metal-organic framework material (MOF), comprising at least one metal center and at least one organic compound as previously described in the context of the present invention.

Advantageously, the metal center of the hydrophobic core is selected from Li, Na, Rb, Mg, Ca, Sr, Ba, Sc, Ti, Zr, Ta, Cr, Mo, W, Mn, Fe, Ru, Os, Co, Ni, Pd, Pt, Cu, Au, Zn, Al, Ga, In, Si, Ge, Sn, Bi, Cd, Mn, Tb, Gd, Ce, La, or Cr, and preferably Cu, Zn, Zr, Ca or Mg. Advantageously, the organic compound is:

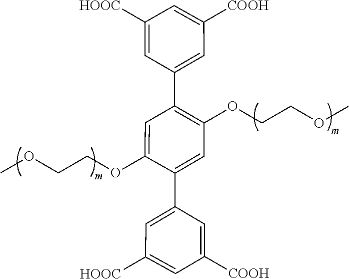

m being an integer number selected from 1 to 7; and the metal center is selected from Cu, Zn, Mg, Ni and Ca.

Advantageously, the organic compound of the MOF is:

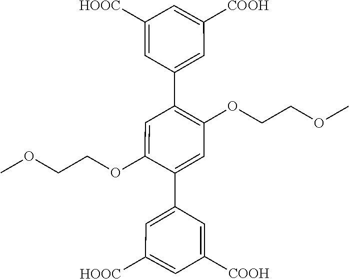

and the metal center is selected from Cu, Zn, Mg, Ni and Ca. Advantageously, the organic compound of the MOF is selected among:

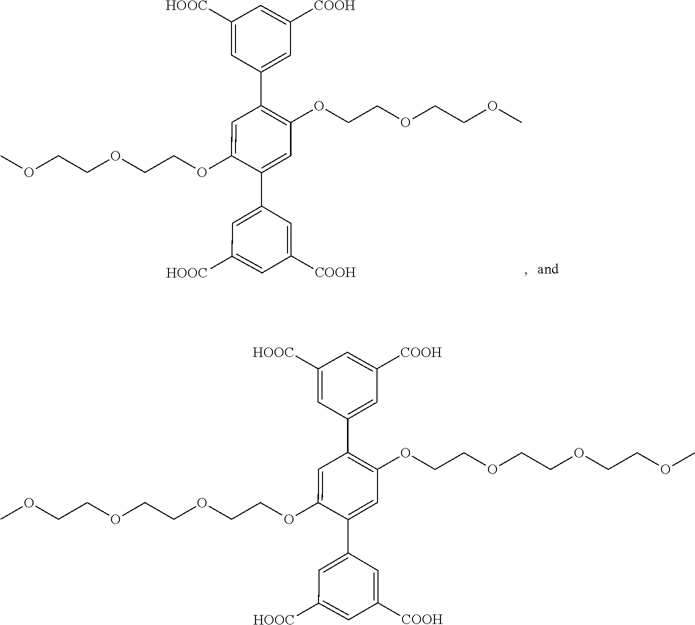

and the metal center is selected from Cu, Zn, Mg, Ni and Ca.

Advantageously, the organic compound is:

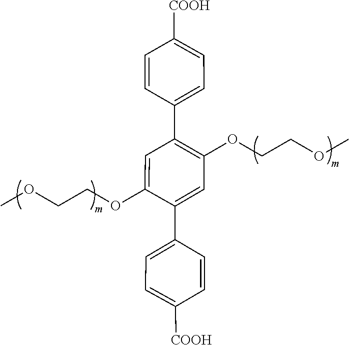

m being an integer number selected from 1 to 7; and the metal center is selected from Cu, Zr, Zn, Fe and Ca.

Advantageously, the organic compound is:

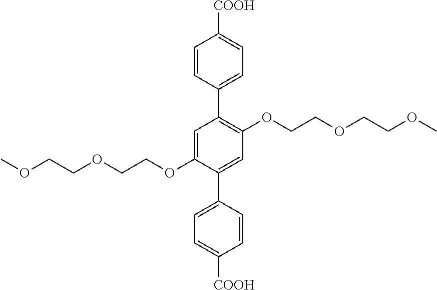

sand the metal center is selected from Cu, Zr, Zn, Fe and Ca.

The present invention relates also to the use of a MOF as previously described in the context of the present invention, for the adsorption of volatile organic compounds (VOCs), and preferably for the adsorption of acetic acid or aldehydes.

Besides, the present invention relates to the use of a MOF as previously described in the context of the present invention, for the decontamination of aqueous solutions containing organic pollutants such as dyes and pharmaceutical compounds, and preferably for the adsorption of a dye, preferably selected among a cationic and an anionic dye, and most preferably selected among at least methylene blue and alizarin yellow R.

In addition, the present invention relates to the use of a MOF as previously described in the context of the present invention, for the adsorption of a gas, preferably selected among methane, hydrogen, acetylene, nitrogen, and CO₂, most preferably CO₂. Said MOF can advantageously be used for:
the separation of gases, for example for treating a gaz mixture containing methane and CO₂;
realizing a gas phase catalysis, such as the preparation of methanol starting to CO.

The concentration of adsorbed gas by the MOF used in the context of the invention for the adsorption of CO₂ is preferably greater than 17 mmol/g at 10 bars, and 0° C., and most preferably greater than 35 mmol/g at 10 bars, and 0° C.

The present invention relates also to a preparation method for the synthesis of a MOF as previously described in the context of the present invention, wherein the metal center is Cu and the MOF is prepared by the dissolution of the organic compound previously described in the context of the present invention, in a DMF/water mixture, followed by an addition plus dissolution of Cu(NO₃)₂.3H₂O into the reaction mixture, the reaction mixture is left at 70-90° C. during several hours in acidic conditions.

Advantageously, the synthesis of the organic compound comprises the steps of:
a—activating phenol groups of a hydroquinone bearing two halogen or two -OTf groups;
b—performing a Suzuki-Miyaura reaction to replace halogen and/or -OTf by aryl groups bearing protected acid acetic substituents; and
c—deprotecting the acid acetic substituents;
preferably according to the following synthetic pathway:

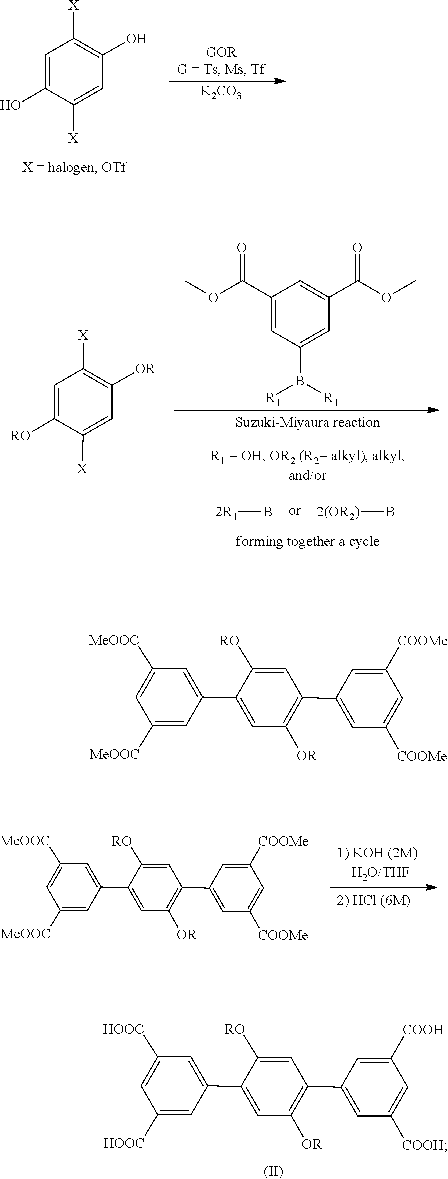

preferably, the X=Br; G=Ts and the first step of the synthetic pathway is realized in DMF at 80° C., and the Suzuki-Miyaura coupling reaction step is performed according to:

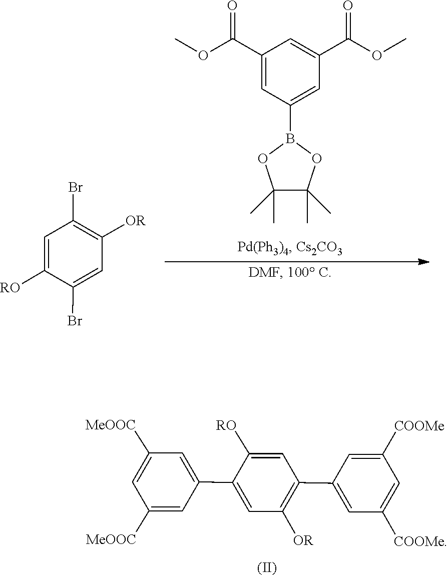

### DEFINITIONS

To facilitate an understanding of the present invention, a number of terms and phrases are defined below.

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties.

As used herein, the term “alkyl”, refers to straight and branched alkyl groups. An analogous convention applies to other generic terms such as “alkenyl”, “alkynyl” and the like. As used herein, “lower alkyl” is used to indicate those alkyl groups (substituted, unsubstituted, branched or unbranched) having about 1-6 carbon atoms. Illustrative alkyl groups include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

In general, the term “aromatic” or “aryl”, as used herein, refers to stable substituted or unsubstituted unsaturated mono-or polycyclic hydrocarbon moieties having preferably 3-14 carbon atoms, comprising at least one ring satisfying Huckle's rule for aromaticity. Examples of aromatic moieties include, but are not limited to, phenyl, indanyl, indenyl, naphthyl, phenanthryl and anthracyl.

As used herein, the term “heteroaryl” refers to unsaturated mono-heterocyclic or polyheterocyclic moieties having preferably 3-14 carbon atoms and at least one ring atom selected from S, O and N, comprising at least one ring satisfying the Hückel rule for aromaticity. The term “heteroaryl” refers to a cyclic unsaturated radical having from about five to about ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Examples of heteroaryl moieties include, but are not limited to, pyridyl, quinolinyl, dihydroquinolinyl, isoquinolinyl, quinazolinyl, dihydroquinazolyl, and tetrahydroquinazolyl.

As used herein, the expression “Cₓ-C_{y}, preferably Cₓ₁-C_{y1}, alkylaryl, aralkyl or aryl”, where x, y, x1 and y1 represent integers denoting the number of carbon atoms in the chemical moiety to which it refers (e.g., “alkylaryl”, “aralkyl”, “aryl”)), means “Cₓ-C_{y}alkylaryl, Cₓ-C_{y}aralkyl or Cₓ-C_{y}aryl, preferably Cₓ₁-C_{y1}alkylaryl, Cₓ₁-C_{y1} aralkyl or Cₓ₁-C_{y1}aryl”. Likewise, the expression “Cₓ-C_{y} alkylaryl, aralkyl or aryl”, means “Cₓ-C_{y}alkylaryl, Cₓ-C_{y}aralkyl or Cₓ-C_{y}aryl”.

As used herein, the term “independently” refers to the fact that the substituents, atoms or moieties to which these terms refer, are selected from the list of variables independently from each other (i.e., they may be identical or the same).

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

### DETAILED DESCRIPTION OF CERTAIN PREFERRED EMBODIMENTS OF THE INVENTION

### Analysis and Experimental Protocols

¹H-NMR and ¹³C-NMR spectra were recorded at 25° C. on Brucker® AV300 (300 MHZ) Bruker AV400 (400 MHZ) or Bruker® AV500 (500 MHz) spectrometers in deuterated solvents with the residual solvent peak used as the internal reference (CDCl₃: 7.26 ppm for ¹H, 77.2 ppm for ¹³C; DMSO-d6: 2.50 ppm for 1H, 49.9 ppm for ¹³C). The abbreviations for specifying the multiplicity of 1H-NMR signals are defined as follows: s=singlet, d=doublet, dd=doublet of doublet, ddd=doublet of doublet of doublets, t=triplet, q=quadruplet, m=multiplet, br=broad. Coupling constants are given in Hertz and chemical shifts in ppm.

MS (Mass spectrometry) was performed at the “Service de Spectrométrie de Masse” of the University of Strasbourg. Low (LRMS) (positive and negative mode ESI: Electro Spray lonization) were recorded on Thermoquest AQA Navigator® with time flight detector.

Elemental analyses were performed on a Thermo Scientific Flash 2000 by the “Service Commun de Microanalyse” of the University of Strasbourg.

UV-Vis spectrometry measurements were performed on a Perkin-Elmer Lambda 900 spectrophotometer in 1 mm quartz cuvettes. Wavelength (A) are given in nm. All solvents used were purchased as spectrometric grades.

Thermo gravimetric analysis (TGA) were performed on a Pyris 6 TGA Lab System apparatus (Perkin-Elmer®), using a N₂ flow of 20 mL/min and a heat rate of 5°/min.

X-Ray diffraction: Single-crystal data were collected on a Bruker® SMART CCD diffractometer with Mo-Kα radiation at 173 K. The structures were solved using SHELXS-97 and refined by full matrix least-squares on F2 using SHELXL-2014 with anisotropic thermal parameters for all non-hydrogen atoms.

The hydrogen atoms were introduced at calculated positions and not refined (riding model). The SQUEEZE command has been employed when disordered solvent molecules were present in structures, to account for the corresponding electron density.

XRPD Diffraction patterns: Powder X-ray diffraction (XRPD) data were recorded using a Bruker® D8 AV diffractometer with Cu-Kα radiation at room temperature. The radiation wavelength λ of the incident X-rays was 1,54 Å and a 20 range is from 4° to 40° was investigated.

BET measurements: Nitrogen adsorption-desorption isotherms were measured at 77K up to 1 bar using the ASAP 2020 Micromeritics® analyzer. Carbon dioxide adsorption-desorption isotherms up to 10 bars at 273K were performed by using ASAP 2050 Micromeritics® analyzer.

The preparation of compounds 1 and 2 were realized according to protocols detailed in [3].

### Synthesis and Characterization of Organic Compounds Used as Ligands in the MOF:

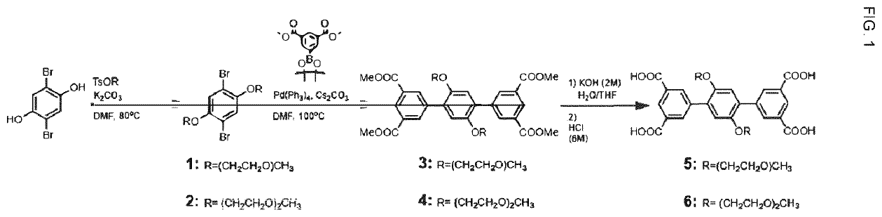

The preparation of the different organic compounds according to formula (II) was performed according to the scheme reported on FIG. 1.
Preparation of TsO (CH₂—CH₂—O)—CH₃, TsO(CH₂—CH₂—O)₂—CH₃, TsO(CH₂—CH₂—O)₃—CH₃, or TsO(CH₂—CH₂—O)₇—CH₃: (for TsO(CH₂—CH₂—O)₂—CH₃, applicable for all) Double necked flask dried oven before reaction. 2-(2-(2-methoxyethoxy) ethoxy) ethanol (9.9 mmol) dissolved in THF (20 mL) and water (15 mL) with NaOH (30 mmol). Later, tosyl chloride (10.5 mmol) dissolved in THF (20 mL) and added dropwise to the mixture while mixing at ambient temperature. Despite THF and water are miscible, in our case (most probably because of NaOH) the layers were separated. After overnight reaction, layers separated. Aqueous layer washed with 2 times with diethyl ether and once with dichloromethane. Organic layer washed twice with water. Organic layers combined, dried on MgSO₄ filtered with cotton and reduced. Pale yellow oil obtained. Yield: 61%.

Alternative method: Double necked flask dried oven before reaction. In an ice bath, 2-(2-methoxyethoxy) ethanol (22 mmol) dissolved in CH₂Cl₂ under Argon atmosphere.

Further, triethyleneamine (22 mmol) poured to the flask. In a separate flask tosyl chloride (23 mmol) dissolved in CH₂Cl₂, white cloudy solution obtained. The solution of tosyl chloride added dropwise to mixture. Reaction continued overnight (18 h). 50 ml water poured to reaction mixture. Aqueous phase washed with dichloromethane, organic phase collected and washed with 3 M HCl (50 ml), NaHCO₃ (50 ml) and with water (50 ml). Dried on MgSO₄, filtered and reduced.

Pale yellow oil obtained and purified further with column (CH₂Cl₂/cyclohexane 1/1). Yield of compounds: ˜80%

Preparation of compounds 1 or 2 or 7: TsO(CH₂—CH₂—O)—CH₃ or TsO(CH₂—CH₂—O)₂—CH₃ (11.22 mmol), dibromohydroquinone (4.48 mmol), and potassium carbonate (26.88mmol) were placed in a double necked bottom flask under inert atmosphere (Ar), and DMF solvent was added. The mixture was stirred overnight (18 h) at 80° C., then the reaction mixture was quenched with 120 ml water to obtain a white precipitate. Small amount of benzoquinone gives brownish color. The product was recrystallized to eliminate this color. Yield for compound 1=53,6%. Compound 2=71%. Compound 7=70%.

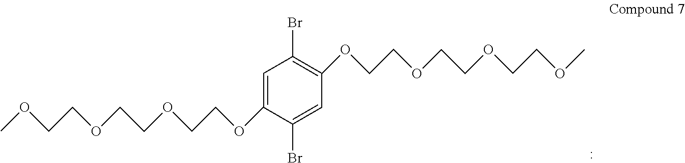

### Alternative Preparation Method for Compound 7:

Double necked flask dried oven before reaction. Dibromohydroquinone (0.25 mmol), compound 3 (0.75 mmol) and potassium carbonate (1 mmol) added to flask. In presence of acetone (50 mL). Then flask evacuated and filled 2 times with Argon. Reaction mixture heated to reflux and gently evacuated and filled 2 times with Argon. The one of the key points of this reaction is prevent the formation of benzoquinone which gives red color to the product even at trace amounts and decreases the yield of the reaction. After 2 days reaction, reaction mixture dried, some water, methanol and excess NaOH added and heated to 50° C. for 5 min while mixing. Later, methanol evaporated, aqueous layer washed twice with diethyl ether. Organic layers combined and washed twice with water. Dried on MgSO4, filtered and reduced. Yield for compound 7: 63%.

### Preparation of Compound 8:

Firstly, dimethyl 5-bromo-benzene-1,3-dicarboxylate (5.4 g, 20 mmol) tared on a triple necked round bottom flask. Under Ar atmosphere, bis-(pinacolato) diborane (6 g, 23.6 mmol) and potassium acetate (oven dried) (5.6 g, 57 mmol) added respectively. Then, dry 1,4-dioxane (50 ml) poured and solution degassed with Ar for 5 min. Finally, Pd(dpff)2Cl2 (0.2 g, 0.27 mmol) added and mixture heated to 80° C. After 24 h reaction stopped and the mixture extracted with ethyl acetate (20 ml). Organic layer dried over MgSO₄, filtered and reduced at vacuum. Obtained crude product purified with column (silica gel, ethyl acetate/petroleum ether, 1/8 v:v). White powder obtained with 85% yield (5.427 g, 17 mmol).

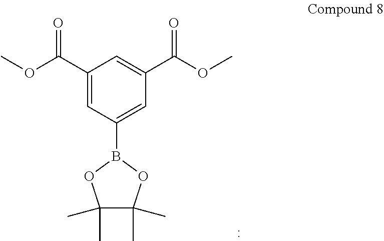

### Preparation of Compound 9:

In a 100 mL flask 3,5-Dimethylphenylboronic acid (3.33 mmol) dissolved in t-BuOH/water (20/15 mL). Later, NaOH (10 mmol) added and mixture heated to reflux. KMnO₄ (27 mmol) added portion wise. After overnight reaction, mixture filtered, t-BuOH evaporated, and acidified. Product filtered and fast rinsed with acetone. Snow white powder obtained. Yield: 60%.

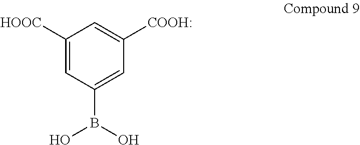

Preparation of compounds 3 or 4: The dimethyl 5-(4,4,5,5-tetramethyl-1,3,2-dioxaborolan-2-yl) isophthalate (6.25 mmol) and compound 1 or 2 (2.08 mmol) were placed in a double necked bottom flask, then 40 ml DMF was added under Argon atmosphere. After 20 min mixing under argon, cesium carbonate (6.25 mmol) and tetrakis (triphenylphosphine) palladium (0) (0.054 mmol) were added, and the mixture was heated under stirring. Reaction continued overnight. Reaction mixture was then dried under reduced pressue, extracted with chloroform, and purified with column. A white solid was obtained.

### Compound 3: Yield=79%;

¹H NMR (500 MHZ, CDCl₃) δ 8.67 (d, J=1.7 Hz, 2H), 8.49 (d, J=1.7 Hz, 4H), 7.07 (s, 2H), 4.13 (t, J=4.7 Hz, 4H), 3.98 (s, 12H), 3.66 (t, J=4.7 Hz, 4H), 3.34 (s, 6H); ¹³C NMR (126 MHZ, CDCl₃) δ 166.38, 150.39, 138.61, 134.90, 130.47, 129.71, 129.44, 116.28, 71.01, 69.32, 59.23, 52.47;

LRMS (ESI⁺) calculated: C₃₂H₃₄O₁₂, 610.21 found: C₃₂H₃₄O₁₂Na, 633.19; elemental analysis (%) for C₃₂H₃₄O₁₂ calculated: C 62.95, H 5.61; found: C 60.52, H 5.54

### Compound 4: Yield=90%;

¹H NMR (500 MHZ, CDCl₃) δ 8.64 (t, J=1.7 Hz, 2H), 8.44 (d, J=1.7 Hz, 4H), 7.02 (s, 2H), 4.12 (t, J=4.9 Hz, 4H), 3.96 (s, 13H), 3.74 (t, J=4.9 Hz, 4H), 3.58-3.53 (m, 4H), 3.46-3.41 (m, 4H), 3.29 (s, 6H).

¹³C NMR (126 MHz, CDCl₃) δ 166.32, 150.28, 138.69, 134.88, 130.45, 129.65, 129.40, 116.06, 71.88, 70.79, 69.68, 69.42, 59.02, 52.47.

LRMS (ESI⁺) calculated: C₃₆H₄₂O₁₄, 698.26, found C₃₆H₄₂O₁₄Na, 721.25; elemental analysis (%) for calculated: C 61.88, H 6.06; found C 58.95, H 5.82.

Preparation of compounds 5 or 6: compound 3 or 4 (1.64 mmol) was dissolved in the mixture of 100 ml THF and 100 ml aq. KOH (2M). The mixture was heated to 90° C. overnight. The heating was stopped, and once reaction mixture reached RT, THF was evaporated under reduced pressure. The aqueous solution was treated with 6M HCl to pH1. The obtained precipitate was filtered and washed with water, plus dried under vacuum overnight.

Compound 5: Yield=89%;

¹H NMR (300 MHZ, DMSO) δ: 13.29 (s, 4H), 8.46 (t, J=1.6 Hz, 2H), 8.42 (d, J=1.6 Hz, 4H), 7.25 (s, 2H), 4.24-4.15 (m, 4H), 3.63-3.54 (m, 4H), 3.22 (s, 6H); ¹³C NMR (126 MHZ, DMSO) δ: 167.12, 150.23, 138.70, 134.67, 131.67, 129.22, 129.08, 116.25, 70.93, 69.07, 58.68;

LRMS (ESI+) calculated: C28H26012,554.14 found: C28H26012, 553.14; elemental analysis (%) for C28H26012 calculated: C 60.65, H 4.46; found: C 52.46, H 4.73.

### Compound 6: Yield=95%;

¹H NMR (500 MHZ, DMSO) δ 13.31 (s, 4H), 8.45 (t, J=1.7 Hz, 2H), 8.38 (d, J=1.6 Hz, 4H), 7.23 (s, 2H), 4.20-4.15 (m, 4H), 3.67-3.62 (m, 4H), 3.46 (dd, J=5.8, 3.8 Hz, 4H), 3.33 (d, J=2.1 Hz, 4H), 3.14 (s, 6H);

¹³C NMR (126 MHZ, DMSO) δ 167.13, 150.24, 138.75, 134.66, 131.62, 129.33, 129.07, 116.33, 71.60, 70.18, 69.48, 69.46, 58.37;

LRMS (ESI⁺) calculated: C₃₂H₃₄O₁₄ 642.19 found: C₃₂H₃₄O₁₄K 681.16 (one K atom plus) elemental analysis (%) for C₃₂H₃₄O₁₄ calculated: C 59.81, H 5.33; found: C 58.13, H 5.18.

Preparation of compound 10 (general method applicable to all final ligands): : Double necked flask dried oven before reaction. Compound 7 (0.107 mmol) and compound 9 (0.24 mmol) tared, then 10 ml ethanol and 5 ml water added under Argon atmosphere. After 20 min mixing under argon, Potassium carbonate (0.47 mmol) and Tetrakis (triphenylphosphine) palladium (0) (0.017 mmol) added and mixture refluxed. Reaction continued overnight.

Reaction mixture filtered over celite. Ethanol from the mixture is evaporated, later some water added following by acidification with concentrated HCl. White slightly yellow solid filtered and rinsed with water and acetone. Yield for compound 10: 52%,

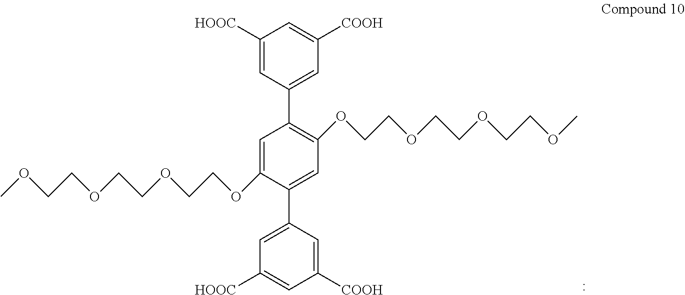

¹H NMR (300 MHZ, DMSO) δ 8.45 (t, J=1.6 Hz, 2H), 8.38 (d, J=1.6 Hz, 4H), 7.23 (s, 2H), 4.17 (dd, J=4.2, 2.2 Hz, 4H), 3.66 (dd, J=5.4, 3.7 Hz, 4H), 3.47-3.45 (m, 4H), 3.38 (d, J=1.0 Hz, 4H), 3.33 (d, J=2.5 Hz, 4H), 3.31 (d, J=1.2 Hz, 4H), 3.17 (s, 6H).

¹³C NMR (126 MHZ, DMSO) δ 167.10, 150.22, 138.76, 134.67, 131.63, 129.28, 129.07, 116.30, 71.61, 70.39, 70.12, 69.92, 69.47, 69.41, 58.42.

LRMS (ESI⁺) for compound 10 calculated: C₃₆H₄₂O₁₆, 730.25; found: C₃₆H₄₁O₁₆, 729.24 Elemental analysis (%) for C36H42016 calculated: C 59.17, H 5.79; found: C 57.39, H 5.63.

Preparation of compounds 11, 12 and 13 (general method applicable to compounds 11-13):

Double necked flask dried oven before reaction. Compound 1 or 2 or 7 (1 eq) and 4-carboxyphenylboronic acid (3 eq) tared, then ethanol and water added (2/1: V/V) under Argon atmosphere. After 20 min mixing under argon, Potassium carbonate (6 eq) and Tetrakis (triphenylphosphine) palladium (0) (0.1 eq) added and mixture refluxed. Reaction continued overnight.

Reaction mixture filtered over celite. Ethanol from the mixture is evaporated, later some water added following by acidification with concentrated HCl. White slightly brown solid filtered and rinsed with water, acetone and diethyl ether. Yield for compound 11: 89%, compound 12: 68%, compound 13: 80%.

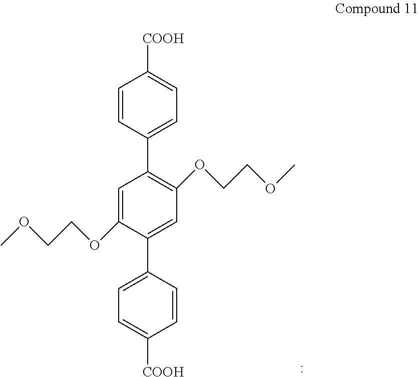

¹H NMR (500 MHZ, DMSO): δ 12.97 (s, 2H), 8.01-7.96 (m, 4H), 7.78 (d, J=8.3 Hz, 4H), 7.15 (s, 2H), 4.19-4.14 (m, 4H), 3.63-3.57 (m, 4H), 3.25 (s, 6H).

¹³C NMR (126 MHZ, DMSO): δ 167.24, 149.80, 142.04, 129.55, 129.42, 129.25, 128.98, 115.93, 70.49, 68.44, 58.23.

LRMS (ESI⁺) for compound 11 calculated: C₂₆H₂₆O₈, 466.16; found: C₂₆H₂₅O₈, 465.16Elemental analysis (%) for C₂₆H₂₆O₈ calculated: C 66.94, H 5.62; found: C 62.88, H 5.30.

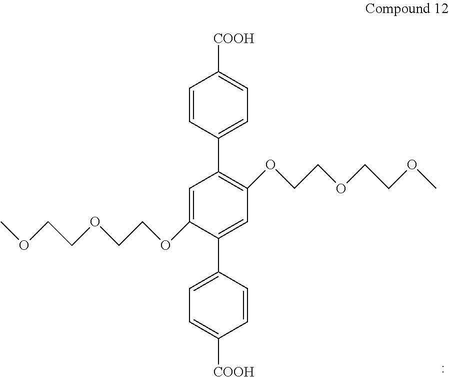

¹H NMR (500 MHZ, DMSO): δ 12.96 (s, 2H), 8.02-7.96 (m, 4H), 7.84-7.78 (m, 4H), 7.17 (s, 2H), 4.20-4.14 (m, 4H), 3.71-3.64 (m, 4H), 3.55-3.49 (m, 4H), 3.44-3.39 (m, 4H), 3.21 (s, 6H).

¹³C NMR (126 MHZ, DMSO): δ 67.17, 149.70, 141.94, 129.51, 129.29, 129.11, 128.88, 115.80, 71.26, 69.61, 68.88, 68.50, 57.99.

LRMS (ESI⁺) for compound 12 calculated: C₃₀H₃₄O₁₀, 554.22; found: C₃₀H₃₄O₁₀K, 593.18 Elemental analysis (%) for C₃₀H₃₄O₁₀ calculated: C 64.97, H 6.18; found: C 64.67, H 6.04.

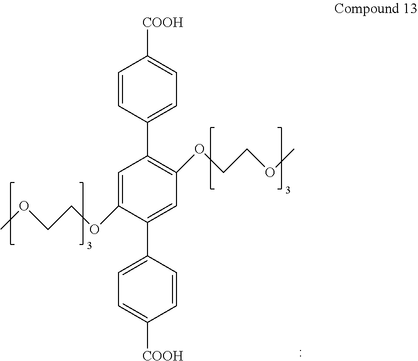

¹H NMR (500 MHZ, DMSO): δ 12.90 (s, 2H), 7.98 (dd, J=8.4, 1.6 Hz, 4H), 7.79 (dd, J=8.3, 1.8 Hz, 4H), 7.16 (s, 2H), 4.17 (dd, J=5.8, 3.4 Hz, 4H), 3.72-3.66 (m, 4H), 3.53-3.51 (m, 4H), 3.50-3.48 (m, 4H), 3.47 (dd, J=5.7, 3.7 Hz, 4H), 3.39-3.38 (m, 4H), 3.19 (s, 6H). (Because of water molecule peaks were slightly “mixed”)

¹³C NMR (126 MHz, DMSO): δ 167.76, 150.21, 131.92, 129.99, 129.82, 129.41, 129.20, 116.30, 71.70, 70.37, 70.32, 70.06, 69.43, 69.01, 58.48.

LRMS (ESI⁺) for compound 13 calculated: C₃₄H₄₂O₁₂ 642.27; found: C₃₄H₄₁O₁₂, 641.26 Elemental analysis (%) for C₃₄H₄₂O₁₂ calculated: C 63.54, H 6.59; found: C 63.90, H 6.45.

Preparation of compounds 14 and 15:

The preparation method is identical to the above described method involving the Tetrakis (triphenylphosphine) palladium (0), the only one difference was the use of dioxane instead of ethanol.

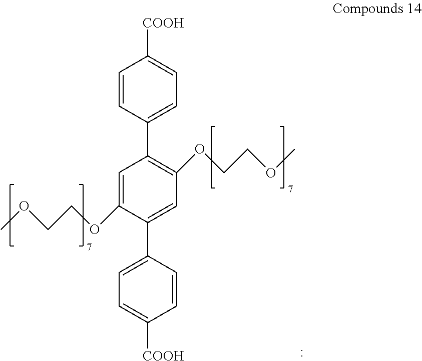

¹H NMR (300 MHZ, DMSO): δ 8.01-7.95 (m, 4H), 7.83-7.77 (m, 4H), 7.16 (s, 2H), 4.17 (t, J=4.6 Hz, 4H), 3.68 (t, J=4.6 Hz, 4H), 3.56-3.45 (m, 48H), 3.22 (s, 6H).

¹³C NMR (126 MHz, DMSO): δ 167.28, 149.80, 129.54, 129.41, 128.96, 128.75, 115.89, 71.27, 69.92, 69.88, 69.77, 69.57, 68.99, 68.62, 58.04./ As the carbons on alkyl chains are similar to each other depending on resolution number of carbon peak on this region (aliphatic) could be different.

LRMS (ESI+) for compound 14 calculated: C₅₀H₇₄O₂₀, 995.12; found: C₅₀H₇₄O₂₀K, 1033.44

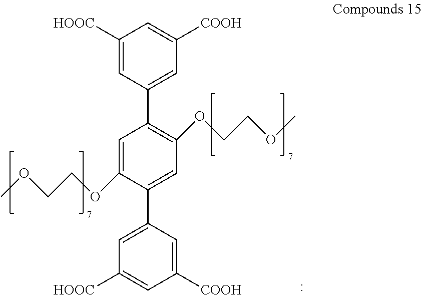

¹H NMR (300 MHZ, DMSO) δ 8.45 (t, J=1.6 Hz, 2H), 8.39 (d, J=1.6 Hz, 4H), 7.23 (s, 2H), 4.20-4.14 (m, 6H), 3.69-3.63 (m, 12H), 3.45 (d, J=4.9 Hz, 38H), 3.2 (s, 6H).

LRMS (ESI+) for compound 15 calculated: C₅₂H₇₄O₂₄, 1083.14; found: C₅₂H₇₄O₂₄K, 1121.42

### Synthesis and Characterization of the MOFs:

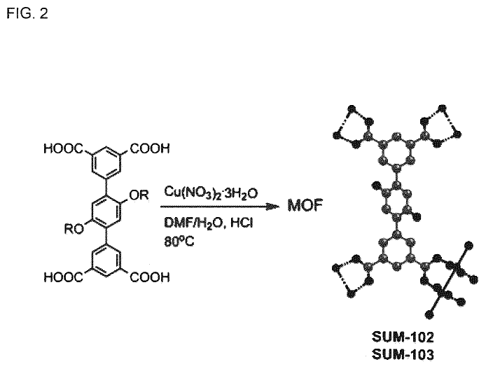

The preparation of the different MOFs starting from the organic compounds according to formula (II) was performed according to the scheme reported on FIG. 2.

SUM-103 and SUM-102: In a 10 ml dram vial, 0.018 mmol ligand (compound 5 or 6) are tared and dissolved in a mixture of 4 ml DMF and 1,3 ml water. Then, 0,072 mmol (17,5mg) of Cu(NO₃)₂.3H₂O was added to the solution and dissolved in the solution. Finally, 33 μl of concentrated HCl was added, the vial is closed and put into dry bath at 80° C. After 24 h, the obtained microcrystalline powder is filtered and washed with DMF to get 14 mg (48% yield) of the MOF SUM-103 and 12.5 mg (45% yield) of the MOF SUM-102.

SUM-403: In a 10 ml dram vial, 0.009 mmol ligand (compound 6) are tared and dissolved in a 2 ml DMF. Then, 0.054 mmol (13.8 mg) of Mg(NO₃)₂x6H₂O was added to the solution and dissolved in the solution. Finally, 2 drops 10 times diluted HCl was added, the vial is closed and put into dry bath at 120° C. After 24 to 48 h, the obtained microcrystalline powder is filtered and washed with.

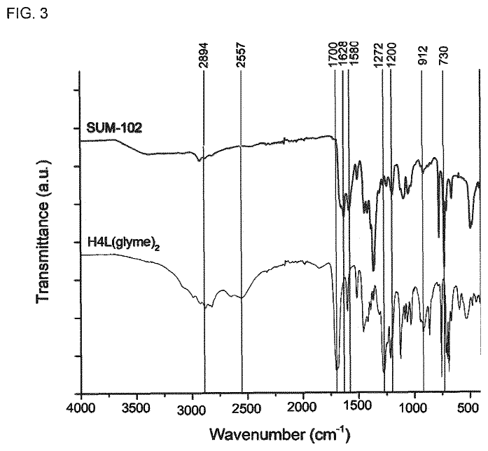

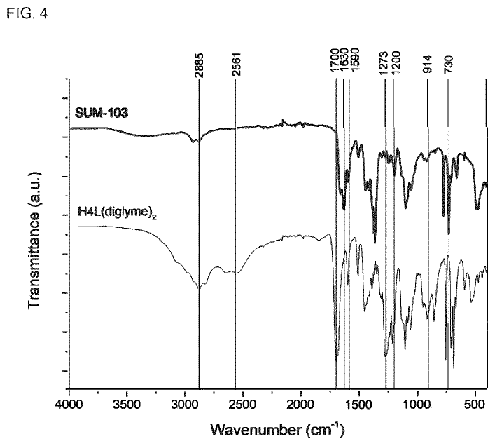

FT/IR spectra of MOFs with their corresponding ligands are shown in FIGS. 3 and 4. (H4L (glyme): is for compound 5; H4L (glyme): is for compound 6).

Since the side chains could not be located by XRD, their presence was established by

FT-IR analysis (see ESI). Indeed, C—H stretching vibrations (2890 cm⁻¹) and the skeletal vibrations of aromatic rings or C—C—O chains (1200 and 1505 cm⁻¹) demonstrated the presence of backbone and side chains.

The formation of MOFs can also be deduced from the C═O stretching band shifted to low energies due to the coordination of the carboxylate groups to Cu (1630 cm⁻¹). Also, Cu—O bond elongation band could be observed at 730 cm⁻¹.

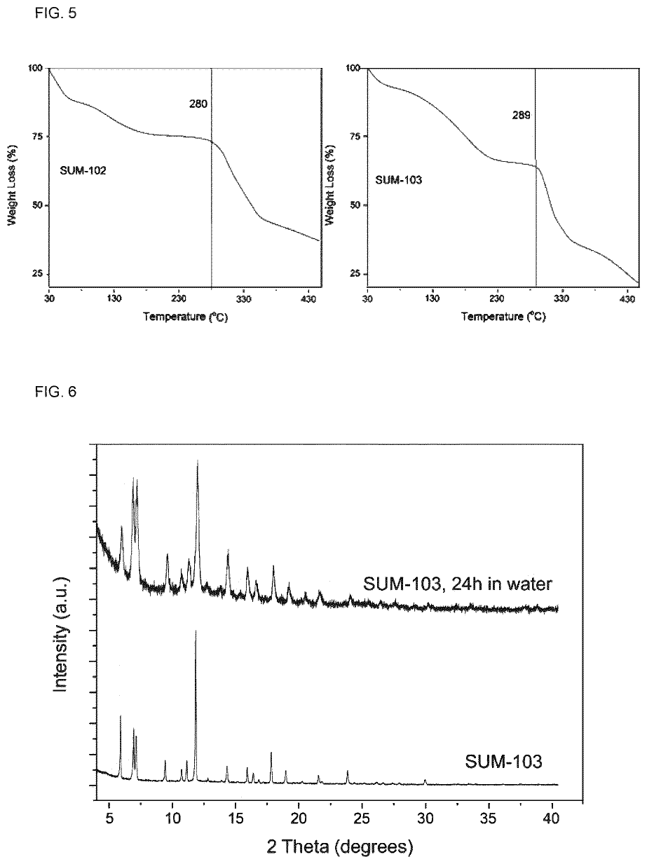

### Thermal Stability (FIG. 5):

TGA analysis up to 450° C. under nitrogen of SUM-102 and SUM-103 are characterized by nearly the same pattern. First phase occurs in two waves of solvents evaporation, one below 50° C. for most volatile solvents and a second one between 70 and 200° C. for less volatile ones. Decomposition of MOFs started at 280° C. and 289° C. respectively. Therefore, addition of oxygen rich side chains does not influence the thermal stability of MOF's.

### Stability in Water of the Two MOFs: SUM-102 and SUM-103 (FIG. 6);

The presence of ethylenoxy units confers to the SUM-102, and more particularly to SUM-103 with two ethylenoxy units, an excellent resistance to water. This after treatment for 8 hours at 160° C. and re-exposure to ambient air, whereas in the case of SUM-102 a loss of crystallinity was observed, SUM-103 appeared to be stable.

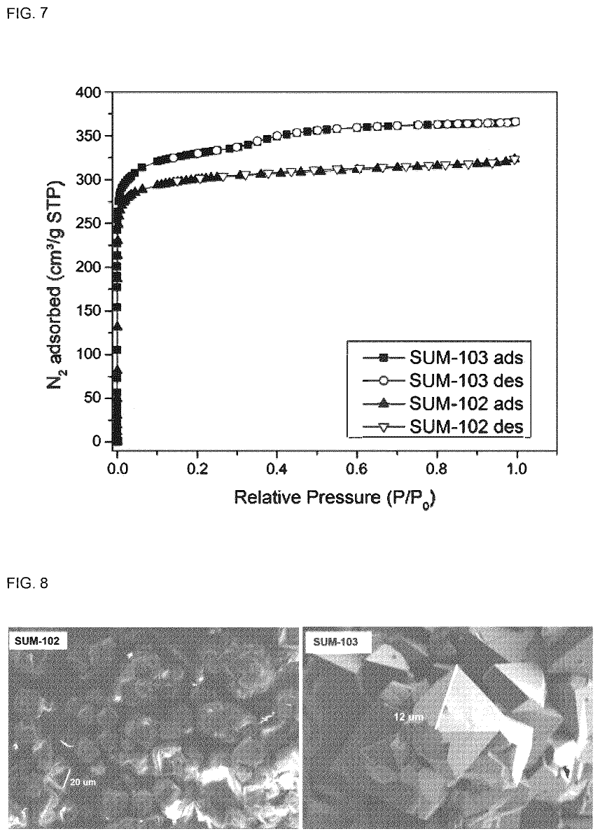

### N₂ Adsorption/BET Surface (FIG. 7);

The nitrogen adsorption of SUM-102 and SUM-103 is displayed on FIG. 7. Despite larger side chains of H4L(diglyme)2 ligand, SUM-103 demonstrates higher surface area. It can therefore be seen that the material with the longest side chains carrying two ethylenoxy units has a greater asorption capacity than the material with only one ethylenoxy unit in the side chains.

The BET surface area measured for SUM-102 are 869 m²/g (N₂) versus 846 m²/g (Ar) and for SUM-103 are 1058 m²/g (N₂) versus 1016 m²/g (Ar) and shows type I isotherm characteristic for microporous materials.

CO₂ adsorption

FIG. 12 shows the CO₂ adsorption by the MOF SUM-103: 38,5 mmol/g adsorbed; 7 mmol/g stays (10 bars, 0° C.).

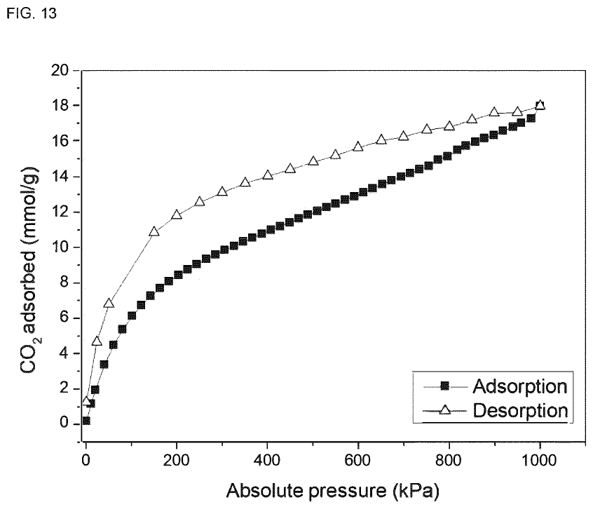

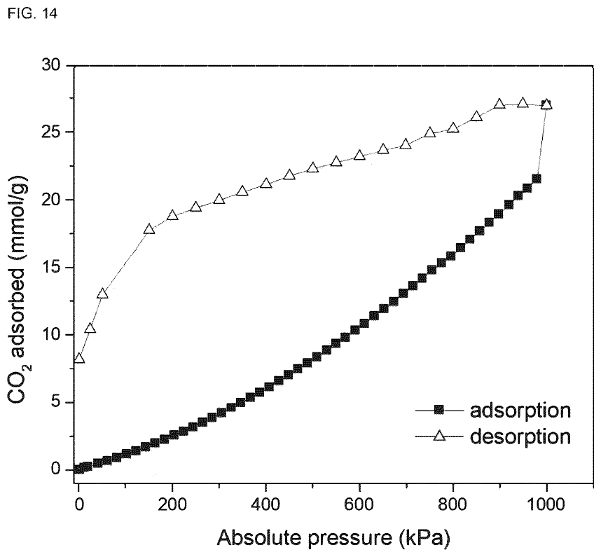

FIG. 13 shows the CO₂ adsorption by the MOF SUM-102: 17 mmol/g adsorbed (10 bars, 0° C.).

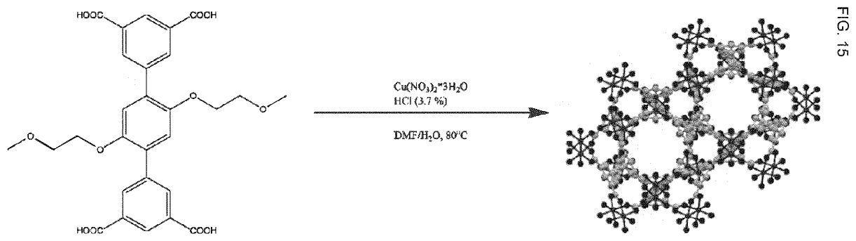

FIG. 14 shows the CO₂ adsorption by the MOF SUM-403: 27 mmol/g adsorbed (10 bars, 0° C.).

### Dye Adsorption

### Methylene Blue Adsorption:

The adsorption capacity of Methylene Blue was calculated based on Equation (1) [4]-[6]. The equilibrium adsorption capacity of adsorbent was calculated using Equation (2) [6].

$$Qt = ( Co - Ct ) \cdot V / m \qquad ( 1 )$$

$$Qe = ( Co - Ce ) \cdot V / m \qquad ( 2 )$$

where Qt and Ct define the adsorption capacity of the adsorbent (mg/g) and the adsorbate concentration (mg/L), respectively. V represents the volume of adsorbate solution and m the mass of MOF adsorbent. Likewise, Qe and Ce define the adsorption capacity of adsorbent and adsorbate concentration (mg/L), respectively, at the equilibrium conditions.

The MB isotherms were fitted with Langmuir and Freundlich models in order to calculate the maximal adsorption capacity and get insights about the nature of the adsorption. Linear form of Langmuir equation is expressed as indicated below:

$$\frac{{c}_{e}}{{Q}_{e}} = \frac{1}{{Q}_{m} {K}_{L}} + \frac{{c}_{e}}{{Q}_{m}} \qquad ( 3 )$$

Ce is the equilibrium concentration, Qe is equilibrium uptake capacity. KL and Qm are obtained from the slope and the intercept of Ce/Qe vs Qe plot. R2 of the linear plot is 0.9992 which shows applicability of this model.

Besides, the separation factor-RL is calculated with Eq (4).

$${R}_{L} = \frac{1}{1 + {C}_{m} {K}_{L}} \qquad ( 4 )$$

Cm is maximal initial concentration of methylene blue. The RL shows favorability of adsorption. The value between 0 and 1 shows good adsorption.

To fit the data to Freundlich model the Eq (5) was used:

$$ln {Q}_{e} = ln {K}_{F} + \frac{1}{n} ln {C}_{e} \qquad ( 5 )$$

To find KF and 1/n (adsorption constants), the plot of InQe vs InCe were drawn. R2 is 0.7507 which doesn't show a good agreement of this model. The values of adsorption constants for both isotherms are summarized in Table 1.

TABLE 1
parameters of isotherm modelling for SUM-103

| Isotherm | | |
|---|---|---|
| model | Constants | |
| Langmuir | Qₘ (mg/g) | 194 |
| | K_{L} (L/mg) | 0.0777 |
| | R_{L} | 0.0605 |
| | R² | 0.9992 |
| Freundlich | K_{F} (mg/g) | 39.1629 |
| | 1/n | 0.3429 |
| | R² | 0.7507 |

In order to properly describe the adsorption process, two popular methods for studying the adsorption kinetics were applied: Pseudo-first order (PFO) and pseudo second order (PSO). Linear equation of PFO (6) and PSO (7) could be expressed as below:

$$ln ( {Q}_{e} - {Q}_{t} ) = ln {Q}_{e} - {k}_{1} t \qquad ( 6 )$$

$$\frac{t}{{Q}_{t}} = \frac{t}{{Q}_{e}} + \frac{1}{{k}_{2} {Q}_{e}^{2}} \qquad ( 7 )$$

Qe and Qt are the amounts of methylene blue adsorbed (mg/g) on MOFs at equilibrium and at the time t. k₁(min⁻¹) and k₂(g/mg·min) are the rate constants of PFO and PSO, respectively.

| Kinetic model | Constants | |
|---|---|---|
| Pseudo First | K₁ (L/min) | 0.0042 |
| Order | Qₑ (mg/g) | 2.29 |
| | R² | 0.9959 |
| Pseudo Second | K₂ (g/mg · min) | 0.0041 |
| Order | Qₑ (mg/g) | 15.21 |
| | R² | 0.9997 |

FIG. 8: Crystals of SUM-102 and SUM-103 are observed under Scanning Electron Microscope. Photographs in FIG. 8 show for SUM-103 size around 10-20 μm, as also observed for SUM-102.

Dyes (methylene blue and alizarin yellow R) adsorption capacity of both MOFs have been evaluated. In case of SUM-103, both isothermal adsorption and kinetic studies were performed. The adsorption parameters have been determined in water (neutral pH) at 30° C. (for isotherm). Both SUM-102 and SUM-103 have been compared under identical conditions. The values of dyes adsorption percentage of dye solutions (10 ml; 10 mg/l) over 3 mg MOF after 48 h and at 30° C. are given in table 2.

TABLE 2
adsorption of dyes versus MOFs sample
(by wt % of dyes adsorbed over MOFs)

| | SUM-102 (%) | SUM-103 (%) |
|---|---|---|
| Alizarin Yellow R | 64 | 77 |
| Methylene blue | 83 | 94 |

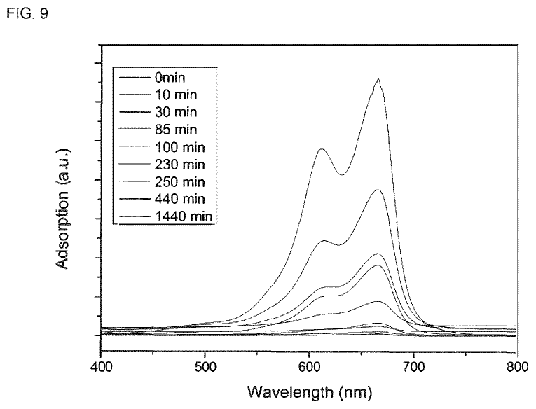

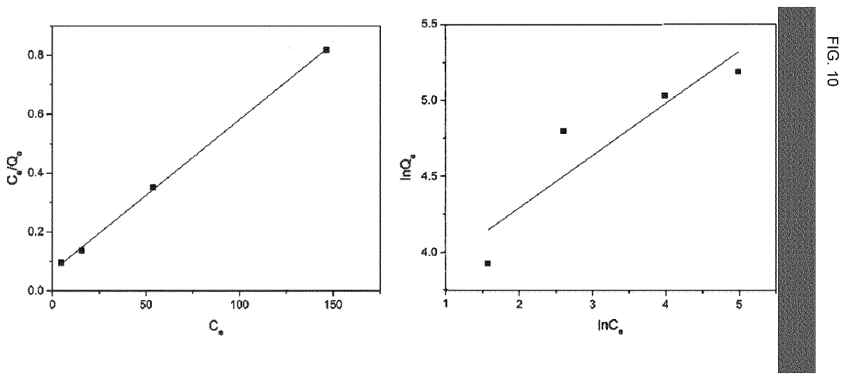

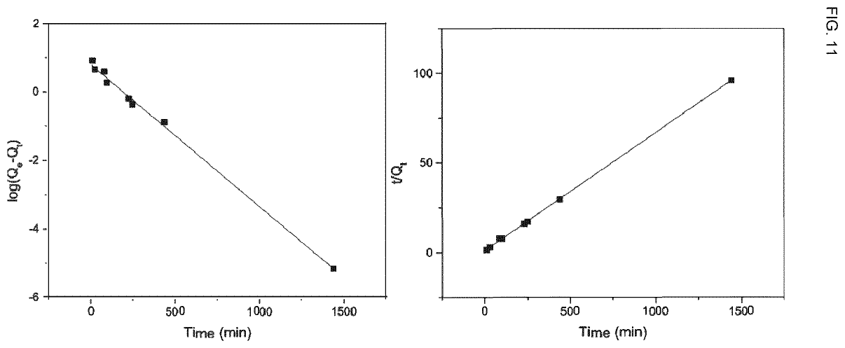

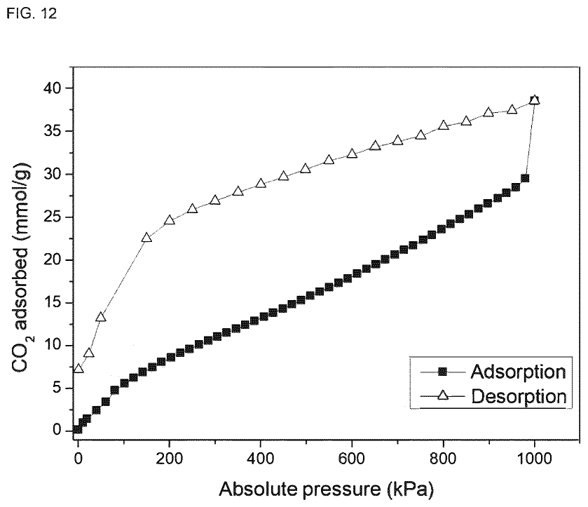

Those values show that methylene blue is more efficiently adsorbed than alizarin yellow R. Also, we observe significant increase in adsorbance with increasing side chains length. The study of the adsorption of methylene blue (MB) on SUM-103 was extended by measurements of isothermal adsorption capacity and a kinetic study, details of these measurements are available in SI. The adsorption protocol was carried out using the batch method by adding 20, 50, 100 and 200 ppm methylene blue solutions over 3 mg MOF in different vials (10 mL) and then placed in the oven at 30° C. After adsorption of methylene blue, the colour of the MOF's changed from blue to deep-blue. Adsorption of methylene blue on MOFs was also assessed by the presence of characteristic strong bands of methylene blue in FT-IR spectrum of final materials (FIG. 9: from left to the right, SUM-103; adsorption kinetics, 2,5 ml, 15 mg/l MB solution after 24 h at room temperature, FT-IR comparison before and after adsorption). After 24 h, the supernatant of methylene blue solution was analysed using a UV-VIS spectrophotometer at 664 nm. FIG. 10 gives Langmuir and Freundlich modelling of MB adsorption of SUM-103. FIG. 11 gives PFO and PSO modelling of MB adsorption of SUM-103 at room temperature.

The isothermal adsorption capacities were modeled according to the models of Langmuir and Freundlich. It is observed that the quantity of methylene blue which binds to the material is 194 mg/g (Langmuir) for SUM-103.

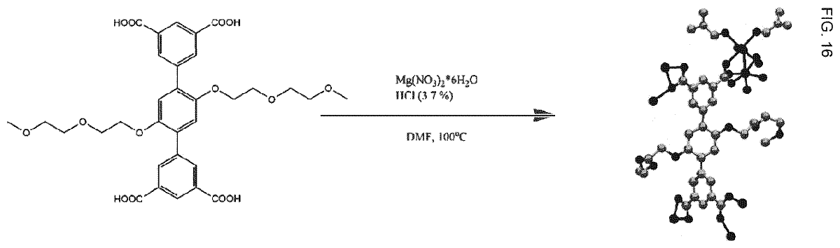

Preparation of SUM-102, SUM-103, and Others MOFs, Starting From the Organic Compounds Being Selected Among the Compounds According to Formula (I) in Connection with the FIGS. 15 to 19:

FIG. 15 shows the network of SUM-102 (or Cu@H4L2 or Cu@10). SUM-103 (n=2) and SUM-104 (n=3) have the same PXRD.

Crystallization conditions: heating of DMF/H₂O solution (4/1.3 mL) of compound 5 (H4L2) (10 mg, 1 eq) and Cu(NO₃)₂*3H₂O (17.9 mg, 4.1 eq) and 33 μL of HCl (12 M) at 80° C. for 24 h.

The same topology crystals were obtained with Zn(NO₃)₂*6H₂O (4.1 eq) with compound 6 (H4L3) (1 eq) in DMF (2 mL).

For CO2 adsorption tests slightly modified protocol was used. But obtained MOF is the same.

TABLE 3

| Chemical formula | C₁₄H₁₅CuO₇ | |
|---|---|---|
| Formula weight | 358.80 g/mol | |
| Temperature | 173(2) K | |
| Wavelength | 0.71073 Å | |
| Crystal system | trigonal | |
| Space group | R −3 m | |
| Unit cell dimensions | a = 18.5565(7) Å | α = 90° |
| | b = 18.5565(7) Å | β = 90° |
| | c = 38.7407(16) Å | γ = 120° |
| Volume | 11552.9(10) Å³ | |
| Z | 2 | |
| Density (calculated) | 0.103 g/cm³ | |
| Absorption coefficient | 0.097 mm⁻¹ | |
| F(000) | 368 | |
| Theta range for data collection | 1.37 to 27.48° | |
| Index ranges | −23 <= h <= 24, −22 <= k <= | |
| | 23, −50 <= l <= 50 | |
| Reflections collected | 36566 | |
| Independent reflections | 3224 [R(int) = 0.0884] | |
| Refinement method | Full-matrix least-squares on F² | |
| Data / restraints / parameters | 3224 / 2 / 107 | |
| Goodness-of-fit on F² | 0.882 | |
| Δ/σₘₐₓ | 0.004 | |
| Final R indices | 2575 data; I > 2σ(I) | R1 = 0.0865, |
| | | wR2 = 0.2479 |
| | all data | R1 = 0.1040, |
| | | wR2 = 0.2680 |

| | |
|---|---|
| Largest diff. peak and hole | 1.440 and −1.076 eÅ⁻³ |

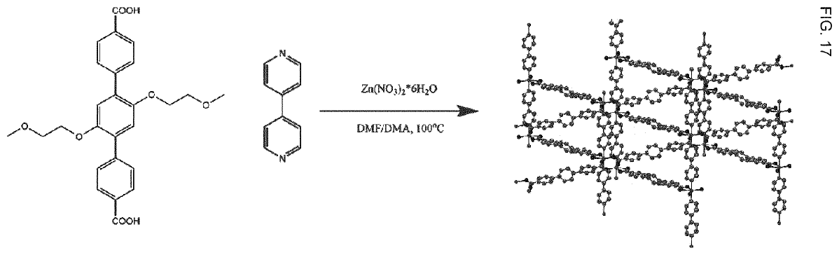

FIG. 16 shows the network of SUM-403 (or Mg@H4L3).

Crystallization conditions: heating of DMF solution (2 mL) of compound 6 (H4L3) (3 mg, 1 eq) and Mg(NO₃)₂*6H₂O (12 mg, 10 eq) and 2 drops of HCl (3.7%) at 100° C. for 24 h.

TABLE 4

| Chemical formula | C₁₉H₂₃MgNO_{8.50} | |
|---|---|---|
| Formula weight | 425.69 g/mol | |
| Temperature | 173(2) K | |
| Wavelength | 0.71073 Å | |
| Crystal size | 0.120 × 0.130 × 0.140 mm | |
| Crystal system | monoclinic | |
| Space group | P 1 21 1 | |
| Unit cell dimensions | a = 10.0803(12) Å | α = 90° |
| | b = 18.2435(18) Å | β = 101.595(5)° |
| | c = 14.2050(17) Å | γ = 90° |
| Volume | 2559.0(5) Å³ | |
| Z | 4 | |
| Density (calculated) | 1.105 g/cm³ | |
| Absorption coefficient | 0.108 mm⁻¹ | |
| F(000) | 896 | |
| Theta range for data collection | 1.46 to 28.13° | |
| Index ranges | −13 <= h <= 13, −23 <= k <= | |
| | 21, −18 <= l <= 18 | |
| Reflections collected | 31265 | |
| Independent reflections | 11883 [R(int) = 0.1269] | |
| Max. and min. transmission | 0.9870 and 0.9850 | |
| Data / restraints / parameters | 11883 / 45 / 659 | |
| Goodness-of-fit on F² | 1.279 | |
| Δ/σₘₐₓ | 4.676 | |
| Final R indices | 6799 data; I > 2σ(I) | R1 = 0.1119, |
| | | wR2 = 0.2495 |
| | all data | R1 = 0.1868, |
| | | wR2 = 0.2840 |

| | |
|---|---|
| Absolute structure parameter | 0.2(3) |
| Largest diff. peak and hole | 0.781 and −0.585 eÅ⁻³ |
| R.M.S. deviation from mean | 0.135 eÅ⁻³ |

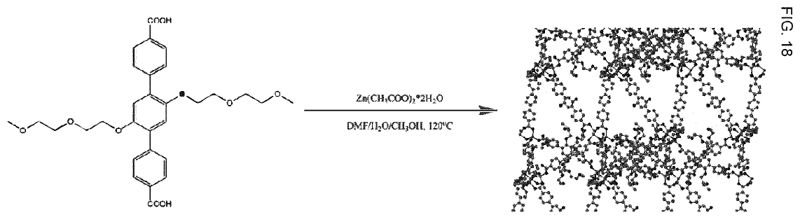

FIGS. 17 shows the Network of SUM-552 (or Zn@H2L2@bipy)

Crystallization conditions: heating of DMF/DMA solution (1/1 mL) of compound 11 (H2L2) (6 mg, 1 eq) and Zn(NO₃)₂*6H₂O (21 mg, 5.5 eq) and 4,4′-bipyridine (3.1 mg, 1.5 eq) at 100° C. for 24 h.

TABLE 5

| Chemical formula | C₃₆H₃₂N₂O₈Zn | |
|---|---|---|
| Formula weight | 686.00 g/mol | |
| Temperature | 173(2) K | |
| Wavelength | 0.71073 Å | |
| Crystal size | 0.150 × 0.180 × 0.190 mm | |
| Crystal system | triclinic | |
| Space group | P −1 | |
| Unit cell dimensions | a = 8.6864(4) Å | α = 73.801(2)° |
| | b = 11.4284(5) Å | β = 83.284(2)° |
| | c = 17.5540(8) Å | γ = 68.094(2)° |
| Volume | 1552.44(12) Å³ | |
| Z | 2 | |
| Density (calculated) | 1.468 g/cm³ | |
| Absorption coefficient | 0.849 mm⁻¹ | |
| F(000) | 712 | |
| Theta range for data | 1.99 to 28.07° | |
| collection | | |
| Index ranges | −11 <= h <= 11, −15 <= k <= | |
| | 15, −23 <= l <= 23 | |
| Reflections collected | 240457 | |
| Independent reflections | 7048 [R(int) = 0.0651] | |
| Max. and min. | 0.7458 and 0.6346 | |
| transmission | | |
| Data / restraints / parameters | 7048 / 9 / 455 | |
| Goodness-of-fit on F² | 1.035 | |
| Δ/σₘₐₓ | 0.001 | |
| Final R indices | 6696 data; I > 2σ(I) | R1 = 0.0370, |
| | | wR2 = 0.0922 |
| | all data | R1 = 0.0392, |
| | | wR2 = 0.0940 |
| Largest diff. peak and hole | 1.163 and −1.173 eÅ⁻³ | |
| R.M.S. deviation from | 0.67⁻³ | |
| mean | | |

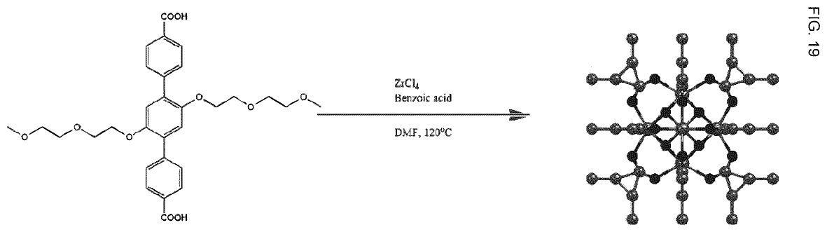

FIG. 18 shows the network of SUM-503 (or Zn@H2L3):

Crystallization conditions: heating of DMF/H₂O/CH₃OH solution (1/1/1 mL) of compound 12 (H2L3) (10 mg, 1 eq) and Zn(CH₃COO)₂*2H₂O (45 mg, 13.5 eq) at 120° C. for 48 h.

TABLE 6

| Formula | C₁₈₀H₃₀N₃₀O₃₀Zn |
|---|---|
| Space group | P −1 |
| Cell lengths | a 18.0412(8) b 20.5164(11) c 28.7012(13) |
| Cell angles | □ 87.969(2) □ 71.835(2) □ 64.132(2) |
| Cell volume | 9019.56 |
| Z | Z: 2 |
| R factor (%) | 15.26 |

FIG. 19 shows the network of SUM-603 (or Zr@H2L3):

Crystallization conditions: heating of DMF (2 mL) solution of compound 12 H2L3 (4 mg, 1 eq) and ZrCl₄ (4.7 mg, 3 eq) and benzoic acid (50 mg, 58 eq) at 120° C. for 24 h.

TABLE 7

| Formula | C₆O₁₆Zr₃ |
|---|---|
| Space group | F m −3 m |
| Cell lengths | a 32.5040(8) b 32.5040(8) c 32.5040(8) |
| Cell angles | □ 90 □ 90 □ 90 |
| Cell volume | 34340.8 |
| Z | 48 |
| R factor (%) | 17.45 |

### REFERENCES

[1] Makal T. A. & al., Tuning the Moisture and Thermal Stability of Metal-Organic Frameworks through Incorporation of Pendant Hydrophobic Groups Crystal growth & Design 2013 13 (11), 4760-4768.

[2] Pasad T. K. & al., Metal-Organic Frameworks Incorporating Various Alkoxy Pendant Groups: Hollow Tubular Morphologies, X-ray Single-Crystal Structures, and Selective Carbon Dioxide Adsorption Properties Chem. Asian J. 2015, 10, 2257-2263.

[3] Vazquez-Molina D. A. & al., Chemical Communications, 2018, 54, 6947-6950.

[4] Feng Y. & al., RSC Adv., 2016, 6, 109608-109612.

[5] Santoso E. & al., Microporous and Mesoporous Materials, 2021, 310, 110620.

[6] Peres E. C. & al., J. Environ. Chem. Eng., 2018, 6, 649-659.

1. Organic compound for the preparation of a MOF, said organic compound being selected among the compounds according to formula (I): 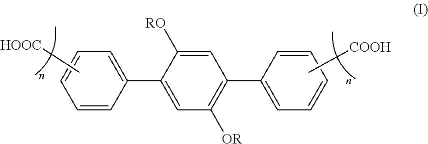
wherein n is from 1 to 2,
R is —(CH₂—CH₂—O)ₘ—CH₃ with m from 1 to 7;
preferably when n=1, the two —COOH groups are located in the para position relatively to the aryl substituted by the two RO groups; and
preferably when n=2, the four —COOH groups are located in the meta position relatively to the aryl substituted by the two RO groups.

2. Organic compound according to claim 1, wherein n is 2; the organic compound being selected among the compounds according to formula (II):

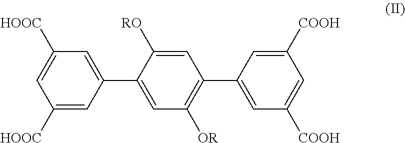

3. Organic compound according to claim 2, wherein m is less than 4.

4. Metal-organic framework material (MOF), comprising at least one metal center and at least one organic compound according to claim 1.

5. Metal-organic framework material according to claim 4, wherein the metal center of the hydrophobic core is selected from Li, Na, Rb, Mg, Ca, Sr, Ba, Sc, Ti, Zr, Ta, Cr, Mo, W, Mn, Fe, Ru, Os, Co, Ni, Pd, Pt, Cu, Au, Zn, Al, Ga, In, Si, Ge, Sn, Bi, Cd, Mn, Tb, Gd, Ce, La, or Cr, and preferably Cu, Zn, Zr, Ca or Mg.

6. Metal-organic framework material according to claim 5, wherein the organic compound is: 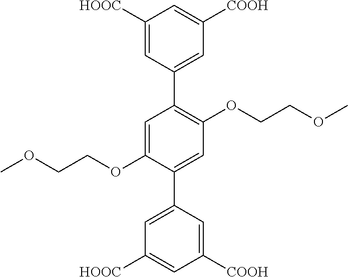
and the metal center is selected from Cu, Zn, Mg, Ni and Ca.

7. Metal-organic framework material according to claim 5, wherein the organic compound is selected among: 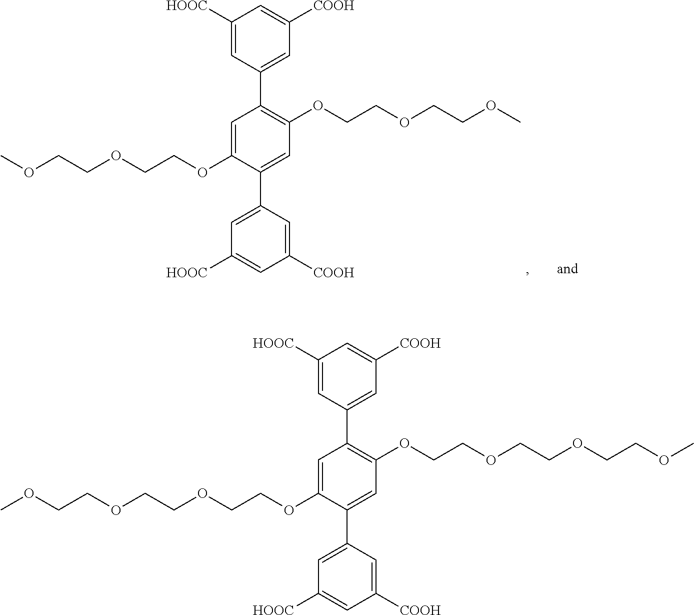
and the metal center is selected from Cu, Zn, Mg, Ni and Ca.

8. Metal-organic framework material according to claim 4, wherein the organic compound is: 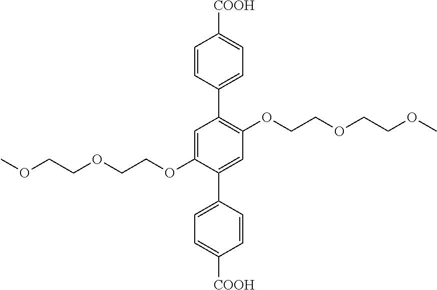
and the metal center is selected from Cu, Zr, Zn, Fe and Ca.

9. Use of a MOF according to 8 claim 4, for the adsorption of volatile organic compounds (VOCs), and preferably for the adsorption of acetic acid or aldehydes.

10. Use of a MOF according to claim 4, for the decontamination of aqueous solutions containing organic pollutants such as dyes and pharmaceutical compounds, and preferably for the adsorption of a dye, preferably selected among a cationic and an anionic dye, and most preferably selected among at least methylene blue and alizarin yellow R.

11. Use of a MOF according to claim 4, for the adsorption of a gas, preferably selected among methane, hydrogen, acetylene, nitrogen, and CO₂, most preferably CO₂.

12. Use of a MOF according to claim 11, for the adsorption of CO₂ wherein the concentration of adsorbed gas is greater than 17 mmol/g at 10 bars, and 0° C.

13. Preparation method for the synthesis of a MOF according to claim 4, wherein the metal center is Cu, and the MOF is prepared by the dissolution of the organic compound, in a DMF/water mixture, followed by an addition plus dissolution of Cu(NO₃)₂.3H₂O into the reaction mixture, the reaction mixture is left at 70-90° C. during several hours.

14. Preparation method for the synthesis of the organic compound according to claim 1, comprising the steps of:
a—activating phenol groups of a hydroquinone bearing two halogen or two -OTf;
groups;
b—performing a Suzuki-Miyaura reaction to replace halogen and/or -OTf by aryl groups bearing protected acid acetic substituents; and
c—deprotecting the acid acetic substituents;
preferably according to the following synthetic pathway:

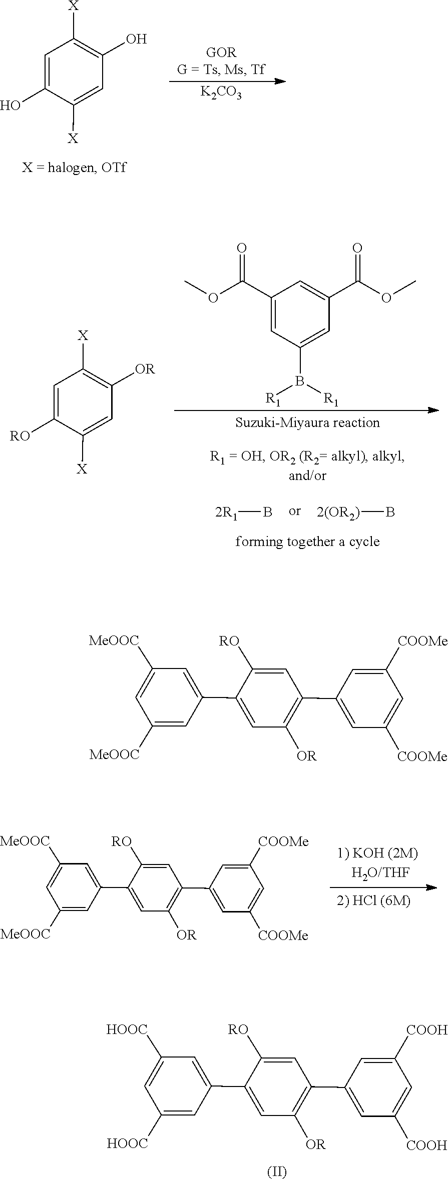

15. Preparation method according to claim 14, wherein X=Br; G=Ts and the first step of the synthetic pathway is realized in DMF at 80° C., and the Suzuki-Miyaura coupling reaction step is performed according to: [chemical structure]
